SEQUENCE LISTING (10 sequences)

SEQ ID NO: 1 - protein, 352 aa, Mus musculus
MAGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSN

SEQ ID NO: 2 - protein, 14 aa, Artificial Sequence
  227-240th amino acid fragment of Oct4 derived from Mus musculus
RTSIENRVRWSLET

SEQ ID NO: 3 - DNA, 1059 bp, Mus musculus
atggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaactga

SEQ ID NO: 4 - protein, 265 aa, Homo sapiens
MHFYRLFLGATRRFLNPEWKGEIDNWCVYVLTSLLPFKIQSQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 5 - protein, 18 aa, Artificial Sequence
  Mouse
ARKRKRTSIENVRRWSLE

SEQ ID NO: 6 - protein, 18 aa, Artificial Sequence
  Bovine
ARKRKRTSIENRVRGNLE

SEQ ID NO: 7 - protein, 18 aa, Artificial Sequence
  Duckbill platypus
ARKRKRTSIENKVRGNLE

SEQ ID NO: 8 - protein, 18 aa, Artificial Sequence
  Human
ARKRKRTSIENVRRGSLE

SEQ ID NO: 9 - protein, 18 aa, Artificial Sequence
  Rhesus macaque
ARKRKRTSIENRVRGSLE

SEQ ID NO: 10 - protein, 15 aa, Artificial Sequence
  Phosphorylated fragment of Mouse Oct4
  MISC_FEATURE (3): Phosphorylation
RTSIENRVRWSLETC

(19) United States
(12) Patent Application Publication
(10) Pub. No.: US 2018/0231568 A1
(43) Pub. Date: Aug. 16, 2018

(54) ANTIBODIES SPECIFIC TO PHOSPHORYLATED OCT4 AND USE THEREOF

(71) Applicant: Seoul National University R&DB Foundation, Seoul (KR)

(72) Inventors: Hong-Duk YOUN, Suwon (KR); Jihoon Shin, Seoul (KR); Hyonchol Jang, Seoul (KR)

(21) Appl. No.: 15/615,938

(22) Filed: Jun. 7, 2017

(30) Foreign Application Priority Data
Feb. 14, 2017 (KR) 10-2017-0020060

(51) Int. Cl. G01N 33/68; C07K 16/18; G01N 33/574

(52) U.S. Cl. CPC G01N 33/6875; C07K 16/18; G01N 2440/14; C07K 2317/34; G01N 2333/47; G01N 33/574

(57) ABSTRACT

Antibodies capable of specifically recognizing phosphorylated Oct4 (Octamer-binding transcription factor 4), and compositions for detecting phosphorylated Oct4 protein or cell cycle, comprising the antibodies specifically recognizing phosphorylated Oct4 protein are provided. The antibodies or the compositions according to the invention may be used to predict characteristic difference between stem cells and cancer stem cells.

### CROSS-REFERENCE TO RELATED APPLICATIONS

This application claims priority to Korean Patent Application No. 10-2017-0020060 filed on Feb. 14, 2017, of which is incorporated by reference herein in its entirety for all purposes.

### TECHNICAL FIELD

This disclosure relates to antibodies specific for phosphorylated Oct4 (Octamer-binding transcription factor 4) and use thereof, particularly to an antibody specifically recognizing a phosphorylated Oct4 or a fragment thereof, a composition for detecting the phosphorylated Oct4 protein or cell cycle comprising an agent of specifically recognizing a phosphorylated Oct4 or a fragment thereof as an active ingredient, and a method of detecting the phosphorylated Oct4 protein or cell cycle, comprising a step of contacting a sample with the agent. The agent or the composition of the present invention may be used to predict difference in characteristics of stem cells or cancer stem cells.

### BACKGROUND OF DISCLOSURE

The activities of transcription factors are directly related to the cell characteristics. In particular, the activities of core transcription factors in embryonic stem cells should be precisely modulated to maintain pluripotency. Oct4, one of the core transcription factors, is an irreplaceable protein in the stem cells and referred to as a master regulator. Recently, it has been known that Oct4 plays an important role in controlling cancer and cancer stem cells.

Accordingly, Oct4 have been continuously studied, and most of the studies have been focused on its transcription activities. Similar to many other transcription factors, the transcription activity of Oct4 is regulated by post translational modification (PTM). In particular, Oct4 S229 residue of Oct4 (SEQ ID NO: 1) is located within the domain which is highly conserved throughout various species. When the S229 residue is phosphorylated, the transcription activity of Oct4 is dramatically weakened.

Also, the transcription activity of Oct4 is regulated by controlling the phosphorylation of Oct4 S229 residue according to cell cycle change. Since the cell cycle of stem cells takes place in a mechanism which is different from the cell cycle of normal cells, the cell cycle of stem cells may not be detected by using existing experimental methods to measure the cell cycle of normal cells. Thus, anti-S229 phosphorylated Oct4 antibodies may be used to detect the cell cycle of stem cells by detecting the phosphorylation of Oct4, and thus, may be applied in stem cell and cancer stem cell studies.

As shown above, S229 residue phosphorylation of Oct4 may be used as a label to show the intracellular transcription activity of Oct4 and the cell cycle of stem cells. However, anti-S229 phosphorylated Oct4 antibodies were not developed, and there were obstacles for various biological, pathological and physiological studies including stem cell and cancer studies. Therefore, there has been an increasing demand to develop the anti-S229 phosphorylated Oct4 antibodies.

### SUMMARY OF DISCLOSURE

An embodiment of the invention is to provide a composition for detecting a phosphorylated Oct4 protein in a sample, comprising an agent for detecting a phosphorylated Oct4 protein as an active ingredient.

Another embodiment of the invention is to provide a composition for detecting a cell cycle, comprising an agent for detecting a phosphorylated Oct4 protein as an active ingredient.

Another embodiment of the invention is to provide an antibody or antigen-binding fragment thereof, specifically binding to a phosphorylated Oct4 or a fragment thereof.

### DETAILED DESCRIPTION OF THE DISCLOSURE

In a detailed embodiment of the invention, the present inventors found that S229 of Oct4 was phosphorylated by expressing Oct4 of SEQ ID NO: 1 in the stem cells, and that S229 (serine at 229^{th} position of amino acid sequence of SEQ ID NO: 1) was highly conserved in various mammals.

In an example of invention, the antibodies specifically recognizing the Oct4 protein phosphorylated at serine 229 are analyzed with western blot and immunoprecipitation, and thus, it is confirmed that the antibodies has an activity to specifically recognize the Oct4 protein phosphorylated at serine 229 of SEQ ID NO: 1. In addition, the amount of Oct4 protein phosphorylated at serine 229 is increased at G2/M phase of stem cells, by analyzing the phosphorylation level of Oct4 S229 of SEQ ID NO: 1 according to the cell cycle of stem cells, using the antibodies specifically recognizing the Oct4 protein phosphorylated at serine 229 of SEQ ID NO: 1.

An embodiment of the present invention is to provide a method of detecting the presence or amount of a phosphorylated Oct4 protein in a biological sample, comprising contacting the sample with an agent of detecting the phosphorylated Oct4 protein or the phosphorylated fragment of Oct 4.

Another embodiment of the invention provide an antibody specifically binding to a peptide comprising an amino acid sequence of SEQ ID NO: 2 with phosphorylated serine at 3^{rd} position (serine 3), but not binding to non-phosphorylated peptide.

Hereinafter, the present invention will be described in more detail.

An embodiment of the invention relates to a composition for detecting (for example, quantitatively detecting) a phosphorylated Oct4 protein in a sample, comprising an agent for detecting a phosphorylated Oct4 protein as an active ingredient. In addition, an embodiment of the invention relates to a composition for detecting (for example, quantitatively detecting) the presence or the amount of a phosphorylated Oct4 protein in a sample, comprising a step of contacting the sample with an agent for detecting a phosphorylated Oct4 protein.

Oct4 protein is also known as POU5F1 protein, and encoded by POU5F1 gene. Oct4 protein is one of the POU family homeodomain transcription factors. Oct4 protein is related to self-replication of undifferentiated embryonic stem cells. The nucleotide sequence of SEQ ID NO: 3 mouse (M. musculus) Oct4 is known as NM_013633.3 (NCBI), and the amino acid sequence of SEQ ID NO: 1 mouse (M. musculus) Oct4 protein is known as NP_038661.2 (NCBI). The nucleotide sequence of human (H. sapiens) Oct4 corresponding to mouse Oct4 protein is provided in SEQ ID NO 4, and known as NP_001272916.1 (NCBI).

The phosphorylated Oct4 protein may refer to Oct 4 protein which may be phosphorylated partially or totally, and preferably, may include phosphorylated serine 229 (S229) in mouse Oct4 protein, but not limited thereto. Serine 229 of mouse (M. musculus) Oct4 protein as set forth in SEQ ID NO: 1 corresponds to serine 236 of human (H. sapiens) (SEQ ID NO: 4) which may be phosphorylated.

The agent for detecting the phosphorylated Oct4 protein in the sample may include chemical small molecules, proteins, peptides, aptamers, nucleic acids (polynucleotides, oligonucleotides, etc.), or antibodies, but not limited thereto.

The antibody as agent for detecting the phosphorylated Oct4 protein or the phosphorylated fragment thereof can specifically bind or recognize the phosphorylated Oct4 protein or the phosphorylated fragment thereof, and preferably, a peptide including phosphorylated serine 229 (S229) in mouse Oct4 protein or the phosphorylated fragment thereof, but not limited thereto. Serine 229 of mouse (M. musculus) Oct4 protein as set forth in SEQ ID NO: 1 corresponds to serine 236 of human (H. sapiens) (SEQ ID NO: 4) which may be phosphorylated.

Specifically, the antibody may specifically recognize a peptide including amino acid sequence of SEQ ID NO: 2 with phosphorylated serine at 3^{rd} position (serine 3). The amino acid sequence of SEQ ID NO:2 corresponds to an amino acid sequence of 14 consecutive amino acids from 227 position to 240 position of SEQ ID NO: 1. The antibody specifically binding to the peptide which essentially including phosphorylated serine 229 of SEQ ID NO: 1, may be a peptide including 14 to 352 consecutive amino acids, 14 to 100 consecutive amino acids or 14 to 50 consecutive amino acids, which essentially includes consecutive 14 amino acids of 227 position to 240 position of full length of Oct4, for examples, mouse Oct4 of SEQ ID NO: 1, but not limited thereto.

The antibody may be a polyclonal antibody obtained by using as an antigen the peptide including an amino acid sequence of SEQ ID NO: 2 with phosphorylated serine at 3^{rd} position. The antibody may be a polyclonal antibody obtained by using the peptide which essentially including phosphorylated serine 229 of SEQ ID NO: 1 and an amino acid 227 to amino acid 240 of SEQ ID NO: 1 as an antigen, but not limited thereto. The antibody is a monoclonal antibody or a polyclonal antibody. The term “polyclonal antibody” denotes herein a mixture of different antibody molecules which react with more than one immunogenic determinant of an antigen.

The antibody is a polyclonal antibody obtained by a method comprising the steps of: a) contacting the sample with a peptide comprising an amino acid sequence of SEQ ID NO: 2 with phosphorylated serine at 3^{rd} position, the peptide which essentially including phosphorylated serine 229 of SEQ ID NO: 1, may be a peptide including 14 to 352 consecutive amino acids, 14 to 100 consecutive amino acids or 14 to 50 consecutive amino acids, which essentially includes consecutive 14 amino acids of 227 position to 240 position of full length of mouse Oct4(SEQ ID NO: 1); and b) collecting the polyclonal antibody.

The antibody specifically binding to the peptide which essentially including phosphorylated serine 229 of SEQ ID NO: 1, may be an antibody obtained by using the peptide which essentially including phosphorylated serine 229 of SEQ ID NO: 1, may be a peptide including 14 to 352 consecutive amino acids, 14 to 100 consecutive amino acids or 14 to 50 consecutive amino acids, which essentially includes consecutive 14 amino acids of 227 position to 240 position of mouse Oct4 full length (SEQ ID NO: 1), or consecutive 14 amino acids of 234 position to 247 position of human Oct4 full length (SEQ ID NO: 4).

The cell cycle at G2/M phase may be selected by analyzing the cells expressing Oct4 S229 phosphorylation signal using the anti-S229 phosphorylated Oct4 antibodies according to the present invention.

As shown in FIG. 2, the antibody or antigen-binding fragment thereof used herein may specifically recognize phosphorylated Oct4 proteins in another species (bovine, cat, chimpanzee, human, mouse, pig, rabbit, rhesus macaque, duckbill platypus, et cetera) in which amino acids adjacent to serine (corresponding to serine 229 of mouse Oct4) are conserved and serine corresponding to serine 229 of mouse Oct4 is phosphorylated.

The “antibody” of the invention is an antibody for the phosphorylated Oct4 which specifically recognizes a specific epitope and binds thereto, and comprises an antigen-binding fragment (antibody fragment) of the antibody molecule as well as a whole antibody. The antibody used herein may be a monoclonal antibody or a polyclonal antibody, and the antibody may be a chimeric antibody or a humanized antibody, but not limited thereto.

The whole antibody includes two full-length light chains and two full-length heavy chains, and each light chain is linked to the heavy chain by disulfide bond. The heavy chain constant region includes gamma (γ), mu (μ), alpha (α), delta (δ) and epsilon (ε) types, which are classified into subgroups of γ1, γ2, γ3, γ4, α1 and α2 type, respectively. The light chain constant region includes kappa (κ) and lamda (λ).

It is well known to one of skill in the art that variants can possess a similar biological activity only where proteins are replaced with amino acids having similar hydropathic index. As disclosed in U.S. Pat. No. 4,554,101, each amino acid residue is assigned the following hydrophilicity values: arginine (+3.0); lysine (+3.0); aspartate (+3.0±1); glutamate (+3.0±1); serine (+0.3); asparagin (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophane (−3.4). Where variations are intended to introduced based on the hydrophilicity value, the substitution is preferably performed between amino acid residues having no more than ±2 difference in hydrophilicity values, more preferably within ±1, much more preferably within ±0.5.

The alteration of amino acid residues without substantially impairing protein activity is well known to one skilled in the art (H. Neurath, R. L. Hill, The Proteins, Academic Press, New York, 1979). Such amino acid alteration includes substitutions of Ala/Ser, Val/Ile, Asp/Glu, Thr/Ser, Ala/Gly, Ala/Thr, Ser/Asn, Ala/Val, Ser/Gly, Thr/Phe, Ala/Pro, Lys/Arg, Asp/Asn, Leu/Ile, Leu/Val, Ala/Glu and Asp/Gly, but not limited thereto.

Considering the afore-mentioned variations having biologically equivalent activities, it could be understood that either antibody of this invention or the nucleic acid encoding the same includes substantially identical sequences to the amino acid sequences set forth in the appended SEQ ID NO: 1 to SEQ ID. NO: 4.

The substantially identical sequences refers to those showing preferably at least 61%, more preferably at least 70%, still more preferably at least 80%, most preferably at least 90% nucleotide similarity to the sequences of the sequences appended herein, as measured using one of the conventionally used sequence comparison algorithms.

The antibody or antigen-binding fragment thereof used herein may be selected from a group consisting of Fab fragment, F(ab′) fragment, F(ab′)₂ fragment, scFv fragment, and (scFv)2 fragment, but not limited thereto.

The term “nucleic acid molecule” used herein comprehensively refers to a DNA (gDNA and cDNA) or RNA molecule, and the basic nucleotides of nucleic acid molecule also include analogues with modified sugar or base as well as natural nucleotides. The sequence of the present nucleic acid molecule encoding the variable regions of heavy and light chain may be modified. Such modification includes addition, deletion or non-conservative or conservative substitution of nucleotide.

The nucleic acids used herein may include a nucleotide sequence showing the substantial homology for the nucleotide sequence. The substantial homology refers to the nucleotide sequence sharing homology of at least 80%, more preferably 90% and most preferable 95% by sequence alignment analysis using maximal alignment between the nucleotide sequence of the invention and other random sequences and algorithm ordinarily known to those skilled in the art.

Another embodiment of the invention relates to a recombinant vector, which includes a nucleic acid molecule encoding a variable domain of heavy chain, a nucleic acid molecule encoding a variable domain of light chain, or both of the nucleic acid molecules. The term “vector” used herein is a tool for expressing a target gene in a host cell, including a plasmid vector; a cosmid vector; and a virus vector such as a bacteriophage vector, an adenovirus vector, a retrovirus vector and an adeno-associated virus vector. A recombinant vector system may be generated by various methods publicly known in the prior arts. The vector used herein may be a conventional cloning vector or an expression vector. Also, the vector used herein may be generated by using prokaryotic cells or eukaryotic cells as a host cell.

The sample may be tissue, cell, whole blood, serum, plasma, saliva, urine, lymph fluid, or spinal fluid, but not limited thereto.

The detection and quantification of the phosphorylated Oct4 protein inside a cell may be performed by western blot. The Besides the method using the western blot analysis, methods such as immunochromatography, immunocytochemistry, immunohistochemistry, enzyme linked immunosorbent assay (ELISA), radioimmunoassay (RIA), enzyme immunoassay (EIA), fluorescence immunoassay (FIA), luminescence immunoassay (LIA), microarray, dot blot, or fluorescence activated cell sorting (FACS) may be used for the detection and quantification of the phosphorylated Oct4 protein inside a cell, but not limited thereto.

The detection of S229 phosphorylated Oct4 protein in the sample may be performed by detecting an antigen-antibody complex formation using a colormetric method, an electrochemical method, a fluorimetric method, a luminometry, a particle counting method, a visual assessment, or a scintillation counting method.

The term “detection” used herein is for detecting an antigen-antibody complex, and may be performed using various label agents such as enzymes, fluorophores, ligands, luminophores, nanoparticles or radioactive materials.

The term “antigen-binding fragment of antibody” refers to any antibody fragment capable of binding to antigen including Fab, F (ab′), F(ab′)2 and Fv, et cetera. Fab has one antigen-binding site which is composed of variable domains of heavy chain and the light chain of the antibody, a constant domain of light chain and the first constant domain (CHI) of heavy chain. Fab′ is different from Fab in that Fab′ has a hinge region containing one or more cysteine residues at C-terminal of CH1 domain of heavy chain. F(ab′)2 antibody is produced by forming a disulfide bond between cysteine residues of hinge region of Fab′. Fv is a minimal antibody fragment composed of variable regions of heavy chain and light chain, and recombinant technique to prepare a Fv fragment is disclosed in PCT WO 88/10649, WO 88/106630, WO 88/07085, WO 88/07086 and WO 88/09344. In two-chain Fv, variable regions of heavy chain and light chain are linked by non-covalent bond, and in single-chain Fv, variable regions of heavy chain and light chain are generally linked by covalent bond via a peptide linker or directly linked to each other at C-terminal, forming a dimer such as two-chain Fv. Such antibody fragments may be obtained using a proteolytic enzymes (e.g., a whole antibody is digested with papain to produce Fab fragments, and pepsin treatment results in the production of F(ab′)₂ fragments), and may be prepared by genetic recombination techniques.

For example, a sample including a lysate of cell expressing Oct4 is analyzed by western blot using an antibodies against Oct4 and antibodies specific for serine 229 (S229)-phosphorylated Oct4 of the present invention, and the western blot signals are detected by Image J (NIH, https://imagej.nih.gov/ij/). A numerical value is a pixel value of each signal. The phosphorylation level of Oct4 existing in each sample may be shown in a numerical value and compared by dividing the numerical value of antibodies specific for the serine 229 (S229)-phosphorylated Oct4 by the numerical value of the Oct4 antibodies.

Enzymes used as a detection label agent include acethylcholinesterase, alkaline postapase, β-D-galactosidase, horse radish peroxidase, and β-latamase, et cetera; fluorophores include fluorescein, Eu3+, Eu3+ chelate or cryptate, et cetera; ligands include biotin derivatives, et cetera; fluorephores include acridium ester and isoluminole derivatives, et cetera; nanoparticles include colloidal gold and colored latex, et cetera; and radioactive materials include 57Co, 3H, 1251, and 1251 Bonton Hunter reagent, et cetera.

According to one embodiment of the invention, an antigen-antibody complex may be detected by using enzyme linked immunosorbent assay (ELISA). ELISA includes various types of ELISA methods, such as a direct ELISA which utilizes a labeled antibody capable of recognizing an antigen attached to a solid support; an indirect ELISA which utilizes a labeled secondary antibody capable of recognizing a capture antibody in an antibody complex which recognizes an antigen attached to a solid support; a direct sandwich ELISA which utilizes another labeled antibody capable of recognizing an antigen from an antigen-antibody complex attached to a solid support; and an indirect sandwich ELISA which includes reacting another antibody with an antigen in an antigen-antibody complex attached to a solid support and then utilizing the labeled secondary antibody capable of recognizing the antibody, et cetera. The antibody of the invention may have a detection label. When the antibody of the invention does not have a detection label, then the antibody of the invention may be captured and detected by treating another antibody which has a detection label.

Another embodiment of the invention relates to a composition for detecting a cell cycle, comprising an agent for detecting a phosphorylated Oct4 protein as an active ingredient, or a method for detecting a cell cycle, comprising contacting the cell with an agent for detecting a phosphorylated Oct4 protein. In addition, an embodiment is to provide a method for detecting cell cycle for cells, comprising contacting the sample with an agent of detecting phosphorylated Oct4 protein.

The agent for detecting the phosphorylated Oct4 protein in the sample may include chemical small molecules, proteins, peptides, aptamers, nucleic acids (polynucleotides, oligonucleotides, etc.), or antibodies, but not limited thereto. The agent, specifically antibody and the detecting method are the same as described above.

Cells to be tested for the cell cycle may include any cells expressing Oct4. For example, the cells may be reproductive cells or somatic cells, and preferably stem cells, but not limited thereto. Cells may include stem cells such as cancer stem cells. The term “cancer stem cell (CSC)” used herein refer to the cell which has a potential to initiate tumor among cancer cells, and is called as tumor initiating cells (TIS), since they are tumorigenic unlike non-tumorigenic cancer cells.

The detectable cell cycle may refer to G2/M phase, but not limited thereto. The G2/M phase of the cell cycle used herein may include G2 phase and M phase of the cell cycle. G2 phase and M phase are status after S phase where DNA synthesis is completed, and thus, the amount of DNA in these phases is doubled compared to the amount of intracellular DNA in GI phase. The term “cell cycle” used herein refers to periodic biochemical nature, which may occur during rapid cell proliferation in the cell culture. Cell cycle used to be divided into mitosis (M phase) and interphase. However, after confirming that DNA replication occurs during a part of interphase (S phase), whole cell cycle is divided into 4 phases, i.e., 3 phases such as GI phase, S phase, and G2 phase in addition to the previous M phase. Thus, the cell cycle indicates a cycle replicating cells where the genetic information is duplicated in S phase and the duplicated genetic information is evenly divided in M phase. Cells proliferate by repeating a continuous process, i.e., growing, dividing, and growing repetitively. In other words, the cell proliferation repeats 4 phases such as M phase (cell division phase, mitosis), G1 phase (first interphase), G2 phase (DNA replication phase), and G2 phase (second interphase), and this whole process refers to as the cell cycle. G1 phase is a phase for preparing DNA synthesis from immediately after mitosis to DNA synthesis initiation, and G2 phase is a phase for preparing mitosis from DNA synthesis completion to mitosis initiation. RNA synthesis or protein synthesis occurs at beginning part of each phase, except M phase. In an embodiment of the invention, S229 phosphorylation of Oct4 may be confirmed by treatment of nocodazole of G2/M arrest drug, and by S229 phosphorylation is disappeared, when G1 phase of the cell cycle is induced by removing the treated nocodazole. Therefore, G2/M phase of cell among the cell cycle may be detected by analyzing an intracellular S229 phosphorylation of Oct4.

The ratio of cells in G2/M phase to total cell number of whole cell population may be calculated by counting numbers of cells showing Oct4 phosphorylation signal, using an antibody specific to the phosphorylated Oct4, and the detection of Oct4 phosphorylation signal may be performed by using the method of detecting or quantifying the intracellular phosphorylated Oct4 protein.

The antibodies of present invention can specifically recognize and bind to the peptide antigens in which serine 229 was phosphorylated, and the peptide antigens which were not phosphorylated did not bind. The antibodies of present invention can specifically recognize the sequence of phosphorylated peptide antigen and specific phosphorylated site to specifically bind.

The antibodies of the present invention are useful as antibodies to detect specifically the phosphorylated form of Oct4 in a biological sample. The antibody or antigen-binding fragment thereof may be an antibody from one of birds, mammals and their eggs, and the mammals may be rabbits, dogs, monkeys, horses, cats, goats, mice, rats, or pigs and the bird may be chicken, but not limited thereto.

### BRIEF DESCRIPTION OF DRAWINGS

FIG. 1 shows the result confirming Oct4 phosphorylation on S229 by Nano-LC-ESI-MS/MS analysis according to an example of the invention.

FIG. 2 shows S229 residue of Oct4, which is conserved in various species.

FIG. 3 is the result showing that an antibody specific to S229 phosphorylated Oct4 specifically recognized Oct4 protein with dot blot according to an example of the invention.

FIG. 4 is the result showing that an antibody specific to S229 phosphorylated Oct4 specifically recognized Oct4 protein phosphorylated at S229 by overexpressing Oct4 WT/S229A/S229D in HEK293T cell and performing western blot according to an example of the invention.

FIG. 5 is the result showing that Oct4 protein phosphorylated at S229 might be specifically immunoprecipitated by using an antibody specific to S229 phosphorylated Oct4 produced according to an example of the invention.

FIG. 6 shows the results detecting the amount of phosphorylated Oct4 according to the cell cycle of E14 embryonic stem cell by western blot, using an antibody specific to S229 phosphorylated Oct4 produced according to an example of the invention.

FIG. 7 shows the results showing immunohistochemical analysis of E14 embryonic stem cells, using an antibody specific to S229 phosphorylated Oct4 produced according to an example of the invention. The images were taken by confocal lasers scanning microscope (magnification: 400×, scale bar: 50 um). The small boxes located below show interphase (1), metaphase (2), or anaphase (3).

FIG. 8 shows the results showing FACS analysis of E14 embryonic stem cells, using an antibody specific to S229 phosphorylated Oct4 produced according to one example of the invention. X axis represents Oct4 signal, and Y axis represents phosphorylated Oct4 signal.

### DETAILED DESCRIPTION OF THE EMBODIMENTS

The invention will now be described in further detail by illustrative examples and embodiments. However, these illustrative examples and embodiments are intended to illustrate the invention more concretely. The scope of the invention should not be limited by these illustrative examples or embodiments.

### REFERENCE EXAMPLE 1

### Culture of HEK293T Cell and Embryonic Stem Cell

HEK (Human embryonic kidney) 293T cells (ATCC) were cultured in DMEM (Hyclone) medium containing 10% (v/v) fetal bovine serum (FBS; Gibco), 100 U/mL penicillin, and 100 ug/mL streptomycin at 37° C. in 5% CO2 incubator.

E14 embryonic stem cells (ESCs, Variant Mouse Regional Resource Centers) and Zhbtc4 embryonic stem cells (Hitoshi Niwa, RIKEN) were cultured in embryonic stem cell culture medium (DMEM (Hyclone), 15% (v/v) FBS (Gibco), 2 mM L-glutamine 55 uM β-mercaptoethanol, 1% (v/v) nonessential amino acids, 100 U/mL penicillin, 100 ug/mL streptomycin (all from Gibco), 1000 U/mL ESGRO (Millipore)) at 37° C. in 5% CO₂ incubator.

### EXAMPLE 1

### Identification of Oct4 S229 Phosphorylation in Embryonic Stem Cells

1-1. Oct4 Overexpression in Stem Cells

To confirm Oct4 phosphorylation in stem cells, Oct4 was overexpressed in Zhbtc4 stem cells (Hitoshi Niwa, RIKEN) in which Oct4 could be removed by doxycycline in the cells, according to the method described below.

In particular, Zhbtc4 stem cells were transfected with pCAG-Flag-Oct4-IRES-PURO plasmids (including Oct4 base sequence of SEQ ID NO: 3) using lipofectamine (Invitrogen), followed by 2 ug/ml puromycin treatment. The cells transfected with the plasmid were selected. The selected cells were treated with 1 μg/ml doxycycline (Sigma, D9891) to overexpress Flag-Oct4 without expressing Oct4 in the cells.

1-2. Identification of Oct4 Phosphorylation Site

To confirm the phosphorylation site of Oct4 expressed in stem cells, the overexpressed Oct4 as shown in 1-1 was analyzed according to method described below.

In particular, the cells overexpressing Flag-Oct4 in 1-1 were lysed using cell lysis buffer (150 mM NaCl, 25 mM Tris-HCl[pH 8.0], 1 mM EDTA, 0.1% (v/v) NP40); Flag-Oct4 was immunoprecipitated with Flag-M2 bead (Sigma, A2220); and Flag-Oct4 was eluted with 3× FLAG Peptide (Sigma, F4799). The purified Flag-Oct4 was treated with Glu-C (Roche) overnight at 37° C. so as to be digested as peptide forms, and then analyzed by Nano-LC-ESI-MS/MS using QTRAP 5500 (AB Sciex). Oct4 protein peptide sequences from the spectrum of peptides obtained from the analysis were selected by using BioWorks BrowserTM (version Rev. 3.3.1 SP1, Thermo).

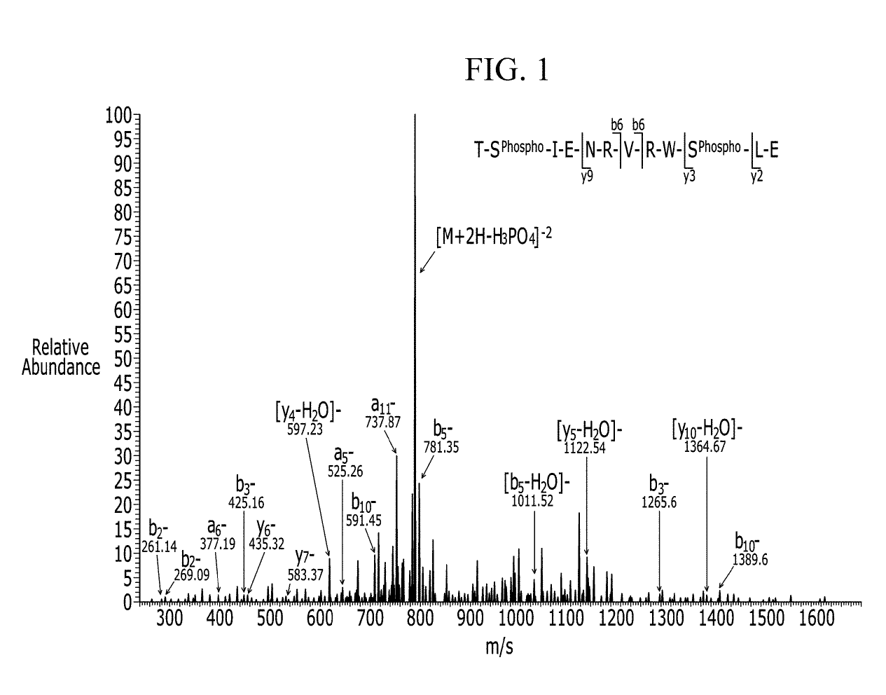

As a result of FIG. 1, a peak in which serine 229 residue of Oct 4 was phosphorylated was identified from the spectrum in which serine 229 residue of Oct4 was observed. It was confirmed that the phosphorylation occurred at serine 229 of Oct4 overexpressed in the stem cells.

1-3. Identification of Interspecies Conservation of Phosphorylated Oct4 Amino Acid Sequence

To confirm whether the adjacent amino acid sequence including phosphorylated amino acid of Oct4 which was newly confirmed in 1-2 was conserved in other species expressing Oct4, the analysis was performed as shown below.

In particular, the homology analysis was performed between sequences including an amino acid corresponding to serine 229 of Mouse (Mus musculus) Oct4 identified above from Oct4 sequences expressed from various mammalian species, such as bovine (Bos taurus), cat (Felis catus), chimpanzee (Pan troglodytes), duckbill platypus (Ornithorhynchus anatinus), human (Homo sapiens), mouse (Mus musculus), pig (Sus scrofa), rabbit (Oryctolagus cuniculus), rhesus macaque (Macaca mulatta), et cetera, by using the alignment function of Uniprot (http://www.uniprot.org/).

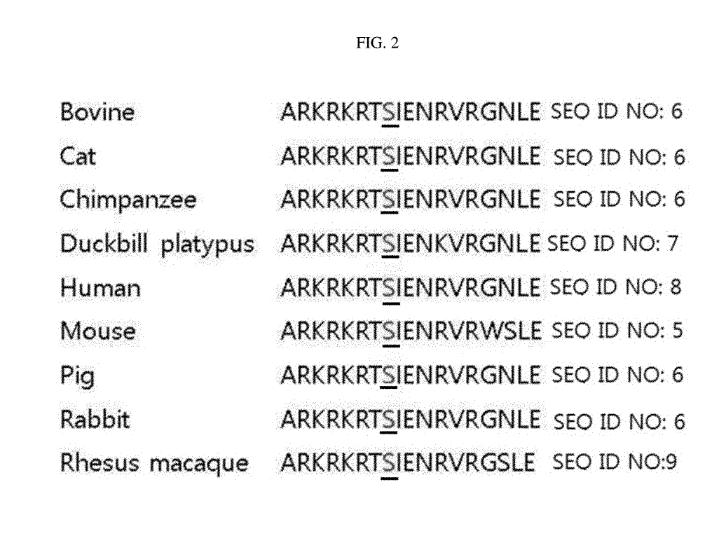

As a result of FIG. 2, it was observed that the amino acid sequence adjacent to serine 229 were conserved in various mammalian species, such as bovine (Bos taurus), cat (Felis catus), chimpanzee (Pan troglodytes), duckbill platypus (Ornithorhynchus anatinus), human (Homo sapiens), mouse (Mus musculus), pig (Sus scrofa), rabbit (Oryctolagus cuniculus), rhesus macaque (Macaca mulatta), et cetera.

### EXAMPLE 2

### Production of Anti-S229 Phosphorylated Oct4 Antibody

2-1. Production of Anti-S229 Phosphorylated Oct4 Antibodies

To produce antibodies recognizing Oct4 S229 phosphorylated protein of Mouse (Mus musculus) (anti-S229 phosphorylated Oct4 antibodies), the experiments shown below were performed.

In particular, phosphopeptide-KLH conjugate antigens consisting of amino acid sequence of ‘RT{pS}IENRVRWSLET(SEQ ID NO: 2)’ from Genscript were produced and injected into 3 rabbits to induce immune reaction. After each rabbit was injected 3 times for immune reaction, sera were collected. Then, a column was produced by attaching the produced antigens to the affinity resins (Genscript), and the antibodies were purified from the collected sera using the column.

2-2. Identification of Activity of Anti-S229 Phosphorylated Oct4 Antibody using ELISA

To confirm if anti-S229 phosphorylated Oct4 antibody produced in 2-1 had an activity of recognizing the Oct4 peptide phosphorylated at serine 229, ELISA was performed as follows.

In particular, 100 μl of phosphorylated antigen peptides and 100 μl of peptides having the same amino acid without phosphorylation, both of which diluted in PBS at a concentration of 4 ug/ml, were added to a 96-well microtiter plate, and coated by incubating for 1 hour at 37° C. Thereafter, each well was blocked by adding 200 μl of 1× blocking buffer (Sigma) and incubating for 1 hour at 37° C. The antibodies produced were sequentially diluted and added in the blocked wells, and incubated at 37° C. for 1 hour to induce binding with the coated antibodies, and then the unbound antibodies were removed by washing three times with PBS.

Hydroperoxidase (HRP) conjugated goat anti-Rabbit Abs (Thermo) were added to the wells in which the purified antibodies and antigens were bound, and incubated at 37° C. for 30 minutes. After binding HRP as described above, the wells were washed with PBS to remove the unbound HRP conjugates. 50 μl of TMB (3,3′,5,5′-tetramethylbenzidene) substrate solution (Sigma, 860336-1G) was added to each well, reacted at room temperature for 10 minutes, and then, the reaction was stopped with 2N sulfuric acid. The resultant reaction degree was obtained by measuring the absorbance at 450 nm using ELISA reader (Perkinelmer, Victor X3).

As a result of Table 1, anti-S229 phosphorylated Oct4 antibodies produced above could specifically recognize Oct4 S229 phosphorylated peptide. The anti-S229 phosphorylated Oct4 antibodies shows higher than 20 times of absorbance at 450 wavelength compared to non-phosphorylated Oct 4, at the dilution rate of 1:100 to 1:32000, higher than 25 times at the dilution rate of 1:100 to 1: 16000, or higher than 30 times at the dilution rate of 1:100 to 1:32000. In other words, the antibody shows 1 or higher, or 1.5 or higher of absorbance at 450 wavelength and the dilution rate of 1:100 to 1:32000, or 2 or higher of absorbance at 450 wavelength and the dilution rate of 1:100 to 1:16000.

TABLE 1

Absorbance (A450)

| Dilution Rate | | Phosphor Peptide | Non-phosphor Peptide |
|---|---|---|---|
| NC | 1:1000 | 0.055 | 0.099 |
| 1 | 1:1000 | 3.027 | 0.088 |
| 2 | 1:2000 | 2.921 | 0.077 |
| 3 | 1:4000 | 2.693 | 0.077 |
| 4 | 1:8000 | 2.380 | 0.075 |
| 5 | 1:16000 | 2.166 | 0.074 |
| 6 | 1:32000 | 1.572 | 0.073 |
| 7 | 1:64000 | 0.887 | 0.072 |
| 8 | 1:128000 | 0.655 | 0.072 |
| 9 | 1:256000 | 0.372 | 0.070 |
| 10 | 1:512000 | 0.221 | 0.069 |
| 11 | Blank | 0.062 | 0.065 |

2-3. Identification of Activity of Anti-S229 Phosphorylated Oct4 Antibody using Dot Blot

To further confirm if anti-S229 phosphorylated Oct4 antibody produced in 2-1 had an activity of recognizing the Oct4 peptide phosphorylated at serine 229, dot blot was performed as follows.

In particular, peptides antigens having the same amino acids as peptide antigens without phosphorylation and phosphorylated peptide antigens were absorbed on the PVDF membrane (Atto) respectively, and incubated in 5% (w/v) skim milk dissolved in PBS for 1 hour at room temperature for blocking. Thereafter, the antibodies produced and purified were incubated at room temperature for 1 hour, and washed with 0.2% (v/v) Triton X-100 dissolved in TBS three times at room temperature for 5 minutes. Then, the antibodies specifically recognizing antigens bound to the PVDF membrane were detected using ECL kit (Thermo Fisher Scientific).

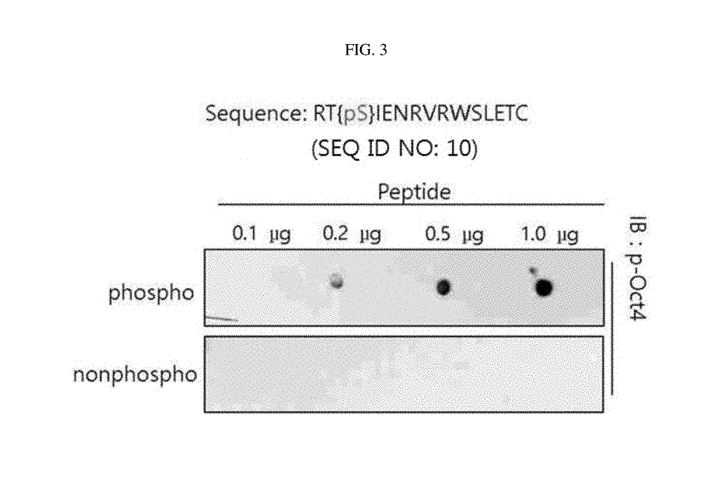

As a result of FIG. 3, it was confirmed that the antibodies produced above could specifically recognize and bind to the peptide antigens in which serine 229 was phosphorylated, and the peptide antigens which were not phosphorylated did not bind. In addition, it was observed that as the amount of phosphorylated peptide antigens absorbed on the PVDF membrane were increased, the amount of anti-S229 phosphorylated Oct4 antibodies was increased proportionally. These results indicated that antibodies produced above could specifically recognize the sequence of phosphorylated peptide antigen and specific phosphorylated site to specifically bind.

### EXAMPLE 3

### Identification of Activity of S229 Phosphorylated Oct4 Protein Recognizing Anti-S229 Phosphorylated Oct4 Antibody

3-1. Western Blot

To confirm if the antibodies produced above could specifically recognize and bind to phosphorylated Oct4 protein as well as phosphorylated peptide, western blot analysis was performed as follows.

In particular, pCAG-Flag-Oct4, pCAG-Flag-Oct4 S229A variant, pCAG-Flag-Oct4 S229D variant expression plasmids were transfected into HEK 293T cells (ATCC) through polyethyleneimine (PEI, Sigma) to overexpress respective proteins in the cells. HEK 293T cells in which respective proteins were overexpressed were treated with a protease inhibitor cocktail kit (P3100-005; GenDEPOT, Barker, Tex.) and a cell lysis buffer containing NaF to lyse cells. Partial sample containing 50 ug of whole cell lysates was separated by SDS-polyacrylamide gel electrophoresis, and transferred to PVDF membrane (Atto) in a transfer buffer (25 mM Tris, 192 mM glycine, 10% [v/v] methanol [pH 8.31) at 80V for 120 minutes. The membrane was blocked by PBS of 5% (w/v) of skim milk containing 0.1% (v/v) of Tween-20 at room temperature for 60 minutes, then incubated with anti-phosphorylated Oct4 (S229), anti-Oct4 (Santacruz), anti-Flag (Sigma), and anti-β-actin (Sigma) antibodies, respectively. HRP-conjugated goat anti-rabbit IgG (1:5000; Thermo) or goat anti-mouse IgG (1:5000; Thermo) was used as secondary antibodies and blots were developed using ECL kit (Thermo Fisher Scientific).

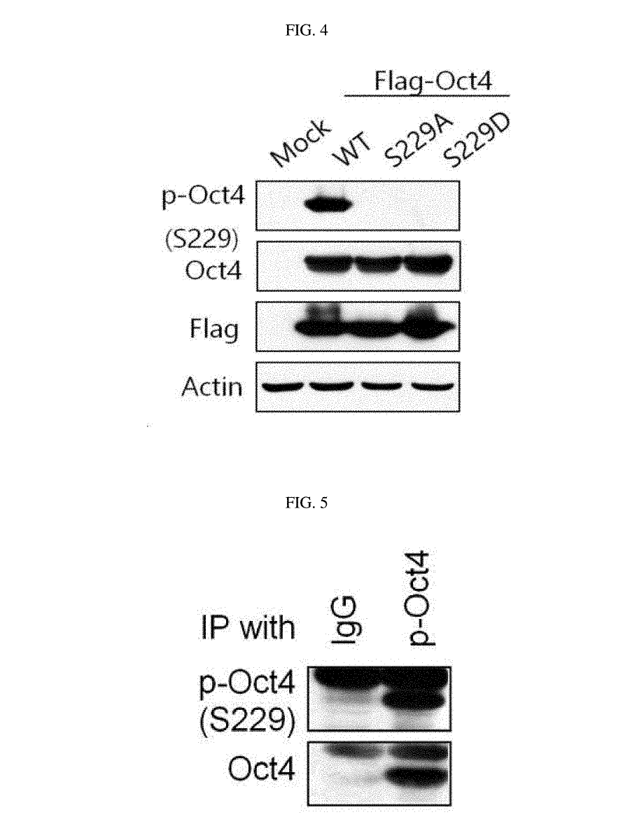

As a result, as shown in FIG. 4, it was confirmed that the anti-S229 phosphorylated Oct4 antibody produced above could not specifically recognize and bind to variant proteins in which serine 229 of Oct4 was substituted by alanine (A) or aspartic acid (D). However, the anti-S229 phosphorylated Oct4 antibody specifically recognized and bound to wildtype Oct4 protein in which serine 229 of Oct4 was not mutated.

3-2. Immunoprecipitation

It was confirmed that anti-S229 phosphorylated Oct4 antibody could be used for immunoprecipitation (IP) analysis. IP analysis was performed as follow.

In particular, E14 embryonic stem cells of Illustrative Example 1 were lysed by a cell lysis buffer (150 mM NaCl, 25mM Tris-HCl[pH 8.0], 1 mM EDTA, 0.1% NP40) containing a protease inhibitor cocktail kit (P3100-005; GenDEPOT, Barker, Tex.) and NaF. Partial samples containing 1 mg of whole cell lysates were incubated with either rabbit IgG (Santacruz) or the antibodies produced above at 4° C. for 4 hours, and then pulled down by protein A plus agarose bead (Santacruz). The pulled down samples were separated by SDS-polyacrylamide gel electrophoresis, and transferred to a nitrocellulose membrane (Amersham, Buckinghamshire, UK) in a transfer buffer (25 mM Tris, 192 mM glycine, 10% [v/v] methanol [pH 8.3]) at 80V for 120 minutes. The membrane was blocked by PBS of 5% (w/v) of skim milk containing 0.1% (v/v) of Tween-20 for 60 minutes at room temperature, and then incubated with anti-phosphorylated Oct4 (S229) antibodies produced above and anti-Oct4 (Santacruz) antibodies, respectively. HRP-conjugated goat anti-rabbit IgG (1:5000; Thermo) or goat anti-mouse IgG (1:5000; Thermo) was used as secondary antibodies, and blots were developed using ECL kit (Thermo Fisher Scientific).

As a result of FIG. 5, it was confirmed that when the whole cell lysates of E14 embryonic stem cells shown in Illustrative Example 1 were immunoprecipitated using the antibodies specifically recognizing S229 phosphorylated Oct4 produced in Example 2 with the comparison group of immunoprecipitation using rabbit IgG antibodies, the S229 phosphorylated Oct4 was more specifically immunoprecipitated compared to the comparison group using rabbit IgG antibodies.

### EXAMPLE 4

### Identification of Oct4 S229 Phosphorylation Pattern according to Cell Cycle of Stem Cells

4-1. Quantitative Analysis of Phosphorylated Oct4 according to Cell Cycle

To quantify the phosphorylation degree of Oct4 S229 according to cell cycle change of stem cells using anti-S229 phosphorylated Oct4 antibodies produced above, E14 embryonic stem cells of Illustrative Example 1 were treated with 200 ng/ml of nocodazole (Calbiochem) for 10 hours. The cell cycle of stem cells treated with nocodazole was arrested at G2/M phase and stayed at G2/M phase without proceeding to G1 phase. However, when nocodazole was removed and the medium was replaced with an embryonic stem cell medium, the cell cycle reached G1 phase after 2 hours, and then, normal cell cycle resumed. The western blot analysis was performed the same as shown in Example 3-1 for E14 embryonic stem cells treated with nocodazole and E14 embryonic stem cells untreated with nocodazole.

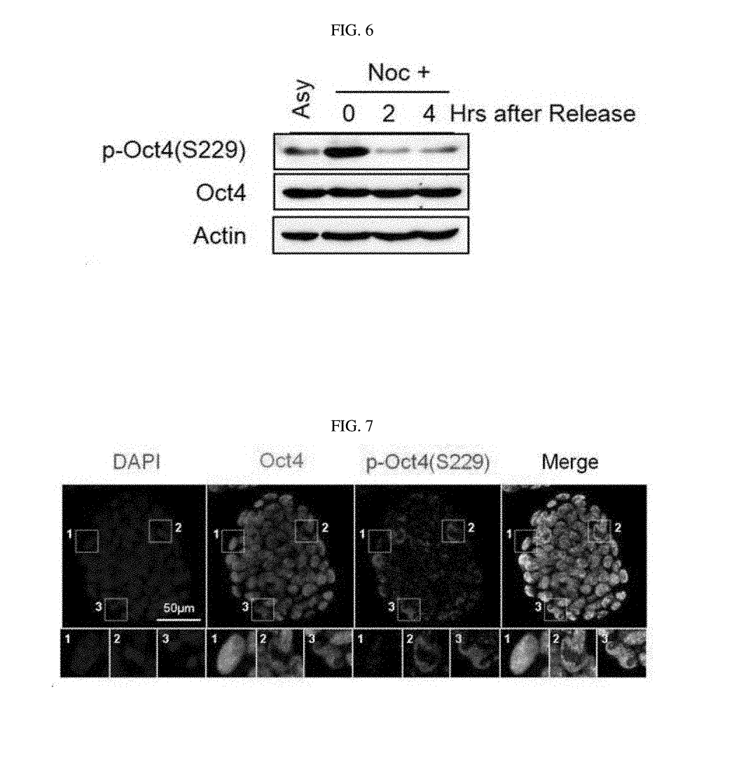

As a result, as shown in FIG. 6, it was confirmed that Oct4 phosphorylation signal was increased in the stem cells which were treated by nocodazole and of which the cell cycle was arrested at G2/M phase. For more precise detection, each signal was digitized by ImageJ as shown in Table 2. The value of phosphorylated Oct4 from the pixel value measured was corrected by dividing by total Oct4 value. As a result, it was confirmed that the value of phosphorylated Oct4 divided by total Oct4 value in the stem cells arrested at G2/M phase was 2.5 times (pixel value) more than the value in the normal stem cells.

TABLE 2

| | Pixel Value of | | | |
|---|---|---|---|---|
| | Phosphorylated | Pixel Value of | Correction | |
| Lane | Oct4 | Oct4 | Value | Ratio |
| 1 | 17575 | 42651 | 0.412072 | 1 |
| 2 | 43360 | 42343 | 1.024016 | 2.48504 |
| 3 | 5858 | 40109 | 0.146053 | 0.354435 |
| 4 | 11257 | 44100 | 0.255265 | 0.619467 |

4-2. Immunocytochemical Analysis

To confirm the phosphorylation change of Oct4 S229 according to cell cycle change of stem cells using anti-S229 phosphorylated Oct4 antibodies produced above, immunocytochemistry (ICC) analysis was performed. ICC was performed by the following method.

The E14 embryonic stem cells of Illustrative Example 1 were cultured on a coverslip, fixed with 4% (v/v) paraformaldehyde, and permeabilized with PBS solution containing 0.5% (w/v) Triton X-100 for 30 minutes at room temperature. Then, the cells were treated with PBS containing 2% (w/v) bovine serum albumin (BSA) for 30 minutes and incubated with anti-Oct4 (Santa Cruz, sc-5279) antibodies diluted at 1:500 and anti-p-Oct4 (S229) antibodies diluted at 1:200. Then, the cells were visualized using Alexa Fluor 488- or 568-conjugated anti-mouse or anti-rabbit antibodies (Invitrogen). The nuclei were counterstained with 4′,6-diamino-2-phenylindole (DAPI). Images were obtained using confocal laser scanning microscope (Carl Zeiss, LSM 510 META).

As shown in FIG. 7, according to immunocytochemical analysis, it showed that S229 phosphorylated Oct4 was not detected in the interphase (1) of the cell cycle, but rather was detected in the middle phase (2) or late phase (3) by the anti-S229 phosphorylated Oct4 antibodies. These results showed that S229 phosphorylation of Oct4 was increased only in stem cells of which the cell cycle was in G2/M phase.

4-3. Flow Cytometry Analysis (FACS)

The phosphorylation degree of Oct4 S229 was determined by flow cytometry analysis in order to detect the cell cycle of stem cells using the anti-S229 phosphorylated Oct4 antibodies prepared above. For flow cytometry analysis, 1×10⁶ of E14 embryonic stem cells of Illustrative Example 1 were collected and fixed with 2% (v/v) paraformaldehyde (Sigma). After washing with PBS twice, the cells were permeabilized with Perm/Wash Buffer (BD, 554723) for 30 minutes. Then, the cells were treated with PBS containing 2% (w/v) bovine serum albumin (BSA) for 30 minutes and incubated with anti-Oct4 (Santa Cruz, sc-5279) antibodies diluted at 1:500 and anti-p-Oct4 (S229) antibodies produced above and diluted at 1:200. Then, they were incubated with Alexa Fluor 488- or 568-conjugated anti-mouse or anti-rabbit antibodies (Invitrogen) and washed with Perm/Wash Buffer (BD, 554723) three times. After that, signals of 10,000 cells were measured using a flow cytometer LSR II (BD).

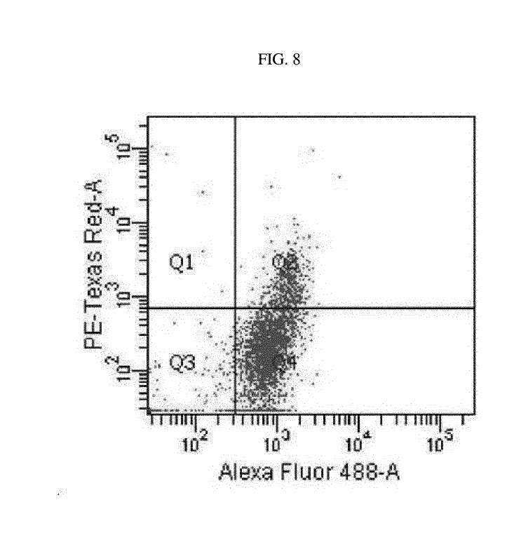

As a result of FIG. 8, the signal of Oct4 (x-axis) and the signal of phosphorylated Oct4 (y-axis) were obtained, and the region from Q1 to Q4 was set according to the intensity of the signal. In particular, the region in which most of cells were present when unstained E14 embryonic stem cells were analyzed by FACS was set as a X-axis region, and the region in which most of cells were present when nocodazole-treated E14 embryonic stem cells were analyzed by FACS was set as a Y-axis region. Thus, E14 embryonic stem cells that normally expressed Oct4 were present only in Q2 and Q4 regions, and the Q2 and Q4 regions were classified according to the intensity of phosphorylated Oct4, respectively.

Table 3 shows the measuring results of cell numbers corresponding to the respective regions shown in FIG. 8. Among them, the region strongly showing the signal of phosphorylated Oct4 is Q2, which corresponds to 13.69% of the total cells. This value is similar to the ratio of stem cells which are in the cell cycle G2/M phase from total stem cells.

Therefore, the cells of which the cell cycle is G2/M phase may be selected by analyzing the cells expressing Oct4 S229 phosphorylation signal with FACS using the anti-S229 phosphorylated Oct4 antibodies prepared above

TABLE 3

| Cell cycle | Cell Number | Percentage |
|---|---|---|
| Q1 | 6 | 0.06 |
| Q2 | 1296 | 13.69 |
| Q3 | 296 | 3.13 |
| Q4 | 7872 | 83.13 |
| [symbol] | 9470 | 100 |

What is claimed is:

1. A method of detecting the presence or amount of a phosphorylated Oct4 protein in a biological sample, comprising contacting the sample with an agent of detecting the phosphorylated Oct4 protein.

2. The method of claim 1, wherein the phosphorylated Oct4 protein includes phosphorylated serine at 229^{th} position (serine 229) in an amino acid sequence of SEQ ID NO: 1.

3. The method of claim 1, wherein the agent is an aptamer, a nucleic acid or an antibody.

4. The method of claim 3, wherein the antibody specifically recognizes a peptide comprising an amino acid sequence of SEQ ID NO: 2 with phosphorylated serine at 3^{rd} position.

5. The method of claim 3, wherein the antibody specifically binds to a peptide having 14 to 352 consecutive amino acids of SEQ ID NO: 1, and an amino acid sequence of SEQ ID NO: 2 with phosphorylated serine at 3^{rd} position as an essential part.

6. The method of claim 3, wherein the antibody is a polyclonal antibody obtained by using a peptide comprising an amino acid sequence of SEQ ID NO: 2 with phosphorylated serine at 3^{rd} position, as an antigen.

7. The method of claim 6, wherein the antibody is a polyclonal antibody obtained by using a peptide which has 14 to 352 consecutive amino acids of SEQ ID NO: 1, and an amino acid sequence of SEQ ID NO: 2 with phosphorylated serine at 3^{rd} position as an essential part, as an antigen.

8. An antibody binding to a peptide comprising an amino acid sequence of SEQ ID NO: 2 with phosphorylated serine at 3^{rd} position, but not binding to non-phosphorylated peptide.

9. The antibody of claim 8, wherein the antibody is a polyclonal antibody obtained by a method comprising the steps of: a) contacting with a peptide comprising an amino acid sequence of SEQ ID NO: 2 with phosphorylated serine at 3^{rd} position.

10. The polyclonal antibody of claim 9, wherein the antibody is a polyclonal antibody obtained by a method comprising the steps of: a) contacting with a peptide comprising an amino acid sequence of SEQ ID NO: 2 with phosphorylated serine at 3^{rd} position, is phosphorylated, and b) collecting the polyclonal antibody.

11. The polyclonal antibody of claim 8, wherein the antibody is a polyclonal antibody obtained by using a peptide which has 14 to 352 consecutive amino acids of SEQ ID NO: 1, and an amino acid sequence of SEQ ID NO: 2 with phosphorylated serine at 3^{rd} position as an essential part, as an antigen.

12. A method for detecting cell cycle phase of cells in a sample, comprising contacting the sample with an agent of detecting phosphorylated Oct4 protein.

13. The method of claim 12, wherein the phosphorylated Oct4 protein includes phosphorylated serine at 229^{th} position (serine 229) in an amino acid sequence of SEQ ID NO: 1.

14. The method of claim 12, wherein the cell cycle is in G2/M phase.

15. The method of claim 12, wherein the agent is an aptamer, a nucleic acid or an antibody.

16. The method of claim 15, wherein the antibody specifically recognizes a peptide comprising an amino acid sequence of SEQ ID NO: 2 with phosphorylated serine at 3^{rd} position.

17. The method of claim 15, wherein the antibody specifically binds to a peptide having 14 to 352 consecutive amino acids of SEQ ID NO: 1, and an amino acid sequence of SEQ ID NO: 2 with phosphorylated serine at 3^{rd} position as an essential part.

18. The method of claim 15, wherein the antibody is a polyclonal antibody obtained by using a peptide comprising an amino acid sequence of SEQ ID NO: 2 with phosphorylated serine at 3^{rd} position, as an antigen.

19. The method of claim 18, wherein the antibody is a polyclonal antibody obtained by using a peptide which has 14 to 352 consecutive amino acids of SEQ ID NO: 1, and an amino acid sequence of SEQ ID NO: 2 with phosphorylated serine at 3^{rd} position as an essential part, as an antigen.

20. The method of claim 12, wherein the sample is tissue, cell, whole blood, serum, plasma, saliva, urine, lymph fluid, or spinal fluid.
